SEQUENCE LISTING (6 sequences)

SEQ ID NO: 1 - protein, 10 aa, Homo sapiens
LLDFVRFMGV

SEQ ID NO: 2 - protein, 9 aa, Artificial
  EBV analogues
ILMEHIHKL

SEQ ID NO: 3 - protein, 8 aa, Artificial
  EBV analogues
DQKDHAVF

SEQ ID NO: 4 - protein, 9 aa, Artificial
  EBV analogues
ALSDHHIYL

SEQ ID NO: 5 - protein, 8 aa, Artificial
  EBV analogues
VITLVYEK

SEQ ID NO: 6 - protein, 9 aa, Artificial
  EBV analogues
SNEEPPPPY

(19) United States
(12) Patent Application Publication
(10) Pub. No.: US 2007/0154953 A1
(43) Pub. Date: Jul. 5, 2007

(54) Identification of antigen epitopes

(76) Inventors: Herwig Brunner, Stuttgart (DE); Gunter Tover, Stuttgart (DE); Thomas Schiestel, Stuttgart (DE); Claudia A. Muller, Tubingen (DE); Thomas Flad, Starzach-Wachendorf (DE)

Correspondence Address: OSTROLENK FABER GERB & SOFFEN, 1180 AVENUE OF THE AMERICAS, NEW YORK, NY, 100368403, US

(21) Appl. No.: 10/547,533

(22) PCT Filed: Mar. 3, 2004
(86) PCT No.: PCT/EP04/02170

§ 371 Date: Aug. 11, 2006

(30) Foreign Application Priority Data
Mar. 5, 2003 (DE) 103 10 261.2

(51) Int. Cl. G01N 33/567; G01N 33/555; G01N 33/53

(52) U.S. Cl. 435/7.2; 436/520; 977/902

(57) ABSTRACT

The present invention relates to methods for identifying and/or detecting T-cell epitopes of a protein antigen, to methods for preparing peptide vaccines against a protein antigen, to methods for controlling the quality of receptor/ligand complexes and/or their components, to methods for preparing nanoparticles having at least one immobilized receptor unit or an immobilized receptor, to methods for preparing nanoparticles having immobilized receptor/ligand complexes, in particular peptide-presenting MHC molecules, to methods for enriching and/or isolating specific CD4⁺-T- or CD8⁺-T-lymphocytes from peripheral blood mononuclear cells, to methods for priming a CD8⁺-T-lymphocyte reaction in vitro, to nanoparticles having an immobilized receptor unit, in particular an immobilized chain of an MHC molecule, to nanoparticles having an immobilized receptor, in particular an immobilized MHC molecule, to nanoparticles having an immobilized receptor/ligand complex, in particular a peptide-presenting MHC molecule, to a peptide vaccine, to a kit for identifying and/or detecting T-cell epitopes of a protein antigen, and to the use of the nanoparticles for identifying and/or detecting T-cell epitopes, for preparing peptide vaccines, for enriching and/or isolating specific T-lymphocytes and for priming a CD8⁺-T-lymphocyte reaction in vitro.

The present invention relates to methods for identifying and/or detecting T-cell epitopes of the protein antigen, to methods for preparing peptide vaccines against a protein antigen, to methods for controlling the quality of receptor/ligand complexes and/or components thereof, to methods for preparing nanoparticles having at least one immobilized receptor unit or an immobilized receptor, to methods for preparing nanoparticles having immobilized peptide-presenting MHC molecules, to methods for enriching and/or isolating specific CD4⁺-T- or CD8⁺-T-lymphocytes from peripheral blood mononuclear cells, to methods for priming and/or restimulating a CD4⁺-T- or CD8⁺-T-lymphocyte reaction in vitro, to nanoparticles having an immobilized receptor unit, in particular an immobilized chain of an MHC molecule, to nanoparticles having an immobilized receptor, in particular an immobilized MHC molecule, to nanoparticles having an immobilized peptide-presenting MHC receptor, to a peptide vaccine, to a kit for identifying and/or detecting T-cell epitopes of a protein antigen, and to the use of the nanoparticles for identifying and/or detecting T-cell epitopes, for preparing peptide vaccines, for enriching and/or isolating specific T-lymphocytes and for priming a CD4⁺-T- or CD8⁺-T-lymphocyte reaction in vitro.

The health of an animal or human organism depends inter alia on how well the organism is capable of protecting itself against pathogenic agents from its environment, or on how well the organism is capable of recognizing and eliminating modified endogenous material. The immune system of the human or animal body, which has these tasks, can be classified into two functional areas, i.e. the innate and the acquired immune system. The innate immune system is the first line of defense against infections, and most potential pathogens are neutralized before they can cause, for example, a noticeable infection. The acquired immune system reacts to surface structures, referred to as antigens, of the intruding organism. There are two types of acquired immune reactions, i.e. the humoral immune reaction and the cell-mediated immune reaction. In the humoral immune reaction, the antibodies present in the bodily fluids bind to antigens, destroying them. In the cell-mediated immune reaction, T-cells capable of destroying other cells are activated. If, for example, proteins associated with a disease are present in a cell, they are, within the cell, fragmented proteolytically to peptides. Specific cell proteins then attach themselves to the antigen or protein fragments formed in this manner and transport them to the surface of the cell, where they are presented to the molecular defense mechanisms, in particular T-cells, of the body.

The molecules which transport the peptides to the cell surface where they are presented are referred to as proteins of the major histocompatibility complex (MHC) . The significance of the MHC proteins is in particular that they enable T-cells to distinguish self antigens from non-self antigens. The MHC proteins are classified into MHC proteins of class I and of class II. The structures of the proteins of the two MHC classes are very similar; however, they differ quite considerably in their function. Proteins of MHC class I are present on the surface of almost all cells of the body. The proteins of MHC class I present antigens which usually originate from endogenous proteins to cytotoxic T-lymphocytes (CTLs). The MHC proteins of class II are only present on B-lymphocytes, macrophages and other antigen-presenting cells. They present mainly peptides which originate from external, i.e. exogenous, antigen sources to T-helper (Th) cells.

MHC molecules of class I are formed constitutively on the surface of almost all cell types within the body. Usually, the peptides bound by the MHC proteins of class I originate from cytoplasmic proteins produced in the healthy host organism itself, which proteins are associated neither with foreign cells nor with degenerated cells. Also, such MHC proteins of class I usually don't stimulate an immune reaction. Accordingly, cytotoxic T-lymphocytes which recognize such self-peptide-presenting MHC molecules of class I are transported into the thymus or, after their release from the thymus, tolerated by the body. MHC molecules are only capable of stimulating an immune reaction when they have bound a non-self peptide to which cytotoxic T-lymphocytes attach themselves. Most cytotoxic T-lymphocytes have, on their surface, both T-cell receptors (TCR) and CD8 molecules. T-Cell receptors are only capable of recognizing non-self peptides and attaching themselves to them if the peptides are present in the form of a complex with the molecules of MHC class I. For a T-cell receptor to be able to bind a peptide/MHC complex, two conditions have to be met. Firstly, the T-cell receptors have to have a structure which permits them to bind themselves to the peptide/MHC complex. Secondly, the CD8 molecule has to attach itself to the α-3 domain of the MHC class I molecules. Each cytotoxic T-lymphocyte expresses a unique T-cell receptor which is only capable of binding a specific MHC/peptide complex.

The peptides attach themselves to the molecules of MHC class I by competitive affinity binding within the endoplasmic reticulum, before they are presented on the cell surface. Here, the affinity of an individual peptide is directly linked to its amino acid sequence and the presence of specific binding motives in defined positions within the amino acid sequence. If the sequence of such a non-self peptide is known, it is possible, for example, to manipulate the immune system against diseased cells using, for example, peptide vaccines. However, the direct analysis of such non-self peptides is difficult owing to a number of factors. For example, very frequently, relevant epitopes, i.e. relevant peptide sequences, are under-represented. What makes it even more difficult is the fact that MHC molecules have a high degree of polymorphism. Thus, in an individual, there may be up to six different polymorphisms among the molecules of MHC class I alone, and in each case, peptide sequences which are in some cases highly different from one another are bound.

Using computer algorithms, it is possible to predict potential T-cell epitopes, i.e. peptide sequences, which are bound by the MHC molecules of class I or class II in the form of a peptide-presenting complex and then, in this form, recognized by the T-cell receptors of cytotoxic T-lymphocytes. This means that the results of such analyses permit the probability of a peptide binding to specific MHC molecules, for example HLA phenotypes, to be predicted. Currently, use is made, in particular, of two programs, namely SYFPEITHI (Rammensee et al., Immunogenetics, 50 (1999), 213-219) and HLA_BIND (Parker et al., J. Immunol., 152 (1994), 163-175). The peptide sequences determined in this manner, which potentially may bind to MHC molecules of class I, then have to be examined in vitro for their actual binding capacity. However, the effectiveness of the methods required for this purpose is highly limited, since only an extremely low number of peptides can be screened and examined simultaneously.

The technical object of the present invention is to provide an improved method for screening potential T-cell epitopes, which method allows 2 simultaneous and rapid examination of a large number of peptide sequences, for example sequences which have already been determined as potential binding partners for specific MHC molecules using computer algorithms, for their capability of binding to specific MHC molecules.

In the present invention, the technical object on which it is based is achieved by providing a method for identifying and/or detecting T-cell epitopes of a protein antigen in vitro, where a population of peptide fragments of the antigen is subjected to competitive binding to a first immobilized receptor unit, preferably and optionally in the presence of a second receptor unit which, together with the first receptor unit, is capable of forming a receptor, where at least one peptide fragment with affinity to the receptor binds to at least the first, preferably both, receptor unit(s), and the bound peptide fragment(s) is/are then isolated and analyzed, comprising
a) immobilization of at least the first receptor unit which has at least one first functional group on a nanoparticle, the surface of which has at least one second functional group which binds the first functional group,
b) production or preparation of a population of peptide fragments of the protein antigen which comprises different sequence ranges of the protein antigen,
c) carrying out a competitive binding of the peptide fragment population to the first receptor unit immobilized on the nanoparticle, optionally, particularly in the case of MHC molecules class II, in the presence of a second receptor unit, where the peptide fragment(s) having affinity to the first receptor unit, or both receptor units, particularly, if present, together with the second receptor unit, binds to the first receptor unit, giving a receptor/peptide fragment complex immobilized on the nanoparticle, and
d) analysis of the immobilized receptor/peptide fragment complex and/or the bound peptide fragment(s).

Thus, in the present invention the technical object on which it is based is achieved by providing a method where a receptor/ligand complex, in particular a receptor/peptide fragment complex is generated in vitro under conditions which substantially correspond to the actual in vivo conditions, for example in a cell containing MHC molecules of class I. Here, according to the invention, a population of peptide fragments is generated which, for example, may represent the complete amino acid sequence of a protein antigen, and the entire peptide fragment population is then, in one step, subjected to binding to an immobilized receptor, in particular an MHC complex, or an immobilized receptor unit, i.e. a chain of the MHC complex. In cases where the receptor is a protein of MHC class I, binding of the peptide fragment(s) to the immobilized first receptor unit, in particular the α-chain, may be sufficient for the identification according to the invention, without a second receptor unit being required. Of course, this second unit may be present nevertheless. This is true in particular for the case where the receptor is a protein of MHC class II. The peptide fragment(s) which has/have affinity to the receptor, to the one receptor unit and/or to both receptor units may then actually form a receptor/ligand complex or a receptor/peptide fragment complex present in immobilized form. Since, according to the invention, the immobilization is on nanoparticles, the resulting receptor/ligand complex can be separated in a simple manner from the peptide fragments which are not capable of forming a receptor/ligand complex, i.e. have no affinity to the first or the two receptor units, since, compared to the peptide fragment bound in the complex, they have no or a considerably lower affinity to the receptor. According to the invention, the peptide fragment or the peptide fragments having affinity can be removed from the population of peptide fragments and analyzed. According to the invention, this peptide fragment can be analyzed in bound form, i.e. as receptor/ligand complex, using, for example, MALDI mass spectrometry. However, according to the invention, it is also possible to separate the peptide fragment bound in the complex from the immobilized complex and to analyze it separately, subjecting it, for example, to sequencing. The peptide fragment population provided for the procedure according to the invention comprises each of the individual peptide fragments in each case in an amount which is sufficient to enable an identification according to the invention.

In the context of the present invention, a “T-cell epitope” is to be understood as meaning a peptide sequence which can be bound by the MHC molecules of class I or II in the form of a peptide-presenting MHC molecule or MHC complex and then, in this form, be recognized and bound by cytotoxic T-lymphocytes or T-helper cells.

In the context of the present invention, a “receptor” is to be understood as meaning a biological molecule or a molecule grouping capable of binding a ligand. A receptor may serve, for example to transmit information in a cell, a cell formation or an organism. The receptor comprises at least one receptor unit and preferably two receptor units, where each receptor unit may consist of a protein molecule, in particular a glycoprotein molecule. The receptor has a structure which complements that of a ligand and may complex the ligand as a binding partner. The information is transmitted in particular by conformational changes of the receptor following complexation of the ligand on the surface of a cell. According to the invention, a receptor is to be understood as meaning in particular proteins of MHC classes I and II capable of forming a receptor/ligand complex with a ligand, in particular a peptide or peptide fragment of suitable length.

A “ligand” is to be understood as meaning a molecule which has a structure complementary to that of a receptor and is capable of forming a complex with this receptor. According to the invention, a ligand is to be understood as meaning in particular a peptide or peptide fragment which has a suitable length and suitable binding motives in its amino acid sequence, so that the peptide or peptide fragment is capable of forming a complex with proteins of MHC class I or MHC class II.

In the context of the present invention, a “receptor/ligand complex” is also to be understood as meaning a “receptor/peptide complex” or “receptor/peptide fragment complex”, in particular a peptide- or peptide fragment-presenting MHC molecule of class I or of class II.

In the context of the present invention, “proteins or molecules of the major histocompatibility complex (MHC)”, “MHC molecules” or “MHC proteins” are to be understood as meaning, in particular, proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for recognizing endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins. The molecules of MHC class II present protein antigens originating from exogenous sources, for example bacterial products. The cellular biology and the expression patterns of the two MHC classes are adapted to these different roles.

MHC molecules of class I consist of a heavy chain of about 45 kDa and a light chain of about 12 kDa and are capable of binding a peptide of about 8 to 10 amino acids if this peptide has suitable binding motives, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin.

MHC molecules of class II consist of an α-chain of about 34 kDa and a β-chain of about 30 kDa and are capable of binding a peptide of about 15 to 24 amino acids if this peptide has suitable binding motives, and presenting it to T-helper cells. The peptide bound by the MHC molecules of class II originates from an exogenous protein antigen. The α-chain and the β-chain are in particular HLA-DR, HLA-DQ and HLA-DP monomers.

In the context of the present invention, a “nanoparticle” is to be understood as meaning a particulate binding matrix which, on its surface, has molecule-specific recognition comprising at least first functional chemical groups. The nanoparticles used according to the invention comprise a core having a surface on which the first functional groups are located, where the first functional groups are capable of binding complementary second functional groups of a molecule in a covalent or non-covalent manner. The molecule, preferably biomolecule, is immobilized on the nanoparticle and/or may be immobilized thereon by interaction between the first and second functional groups. The nanoparticles used according to the invention have a size of <500 nm, preferably <150 nm. The core of the nanoparticles preferably consists of chemically inert inorganic or organic materials, particularly preferably of silicon dioxide.

In the context of the present invention, the “first functional group” is to be understood as meaning a chemical group present in a receptor unit, in particular a chain of an MHC molecule, which group is capable of interacting with a complementary functional group present, for example, on the surface of the nanoparticle in such a manner that an affinity bond, preferably a covalent bond, may be formed between the two binding partners. According to the invention, it is envisaged that the first functional group is selected from the group consisting of carboxyl groups, amino groups, thiol groups, biotin groups, His tag, FLAG tag, Strep tag I groups, Strep tag II groups, histidine tag groups and FLAG tag groups.

According to the invention, the second functional group, i.e. the functional group on the surface of the nanoparticle, is selected from the group consisting of amino groups, carboxyl groups, maleinimido groups, avidin groups, streptavidin groups, neutravidin groups and metal chelate complexes.

Thus, a nanoparticle used according to the invention has, on its surface, at least one second functional group which is attached covalently or non-covalently to a first functional group of a receptor unit, where the first functional group is a different group from the second functional group. The two groups binding to one another have to be complementary to one another, i.e. capable of forming a covalent or non-covalent bond with one another.

If, according to the invention, the first functional group used is, for example, a carboxyl group, the second functional group on the surface of the nanoparticles is an amino group. If, according to the invention, conversely an amino group is used as first function group of the receptor unit, according to the invention, the second functional group on the nanoparticle surface is a carboxyl group. If, according to the invention, a thiol group is selected as first functional group of the receptor unit, according to the invention, the second functional group is a maleinimido group. If, according to the invention, biotin groups and/or Strep tag I groups and/or Strep tag II groups are used as first functional groups of the receptor unit, the second functional group on the nanoparticle surface is an avidin group and/or a streptavidin group or a neutravidin group. If, according to the invention, the first functional group of the receptor unit used is a thiol group, the second functional group on the nanoparticle surface is a maleimido group.

The above-mentioned first and/or second functional groups may be attached to the immobilizing receptor unit and the surface of the nanoparticles, respectively, with the aid of a spacer, or they can be introduced on the nanoparticle surface or into the receptor unit using a spacer. Thus, on the one hand, the spacer serves to keep the functional group at a distance from the nanoparticle surface or the receptor unit, on the other hand, it serves as carrier for the functional group. According to the invention, such a spacer may be alkylene groups or ethylene oxide oligomers having 2 to 50 carbon atoms which, in a preferred embodiment, is substituted and has heteroatoms. The spacer may be flexible and/or linear.

In a preferred embodiment of the invention, it is envisaged that the first functional groups are a natural component of the receptor unit. In a further preferred embodiment of the invention, it is envisaged that the first functional groups are introduced into the receptor unit using genetic engineering, biochemical, enzymatic and/or chemical derivatization or chemical synthesis. Unnatural amino acids, for example, can be introduced into the receptor unit using genetic engineering or during a chemical protein synthesis, for example together with spacers or linkers. Such unnatural amino acids are compounds having an amino acid function and a radical R which are not defined by the naturally occurring genetic code and these amino acids preferably have a thiol group. According to the invention, it may also be envisaged to modify a naturally occurring amino acid, for example lysine, for example by derivatizing its side chain, in particular its primary amino group, with the carboxylic acid function of levulinic acid.

In a further preferred embodiment of the present invention, functional groups may be introduced into the receptor unit by modification, where tags, i.e. markers, have to be added to the receptor unit, preferably to the C-terminus or the N-terminus. However, these tags may also be positioned intramolecularly. In particular, it is envisaged that a protein receptor is modified by adding at least one Strep tag, for example a Strep tag I or Strep tag II or biotin, for example via BirA. According to the invention, Strep tags are also to be understood as meaning functional and/or structural equivalents, if they are capable of binding streptavidin groups and/or equivalents thereof. According to the invention, the term “streptavidin” thus also embraces its functional and/or structural equivalents.

According to the invention, the surface of the nanoparticle is characterized in that it is modified by application of the complementary second functional groups which bind the first functional groups. According to the invention, it is, in particular, envisaged that the functional groups are applied to the nanoparticle surface using standard processes, such as graft polymerization, silanization, chemical derivatization and similar suitable processes.

In a preferred embodiment of the invention, it is envisaged that the nanoparticle surface can be modified by applying additional functionalities.

In a preferred embodiment, the surface of the nanoparticles may have chemical compounds which prevent or reduce unspecific adsorption of other proteins on the nanoparticles. With particular preference, the surface has ethylene glycol oligomers.

According to the invention, it is also possible to anchor, separately or additionally, ion exchange functions on the surface of the nanoparticles. This applies in particular to the case where the analysis of the receptor/ligand complex obtained, in particular the peptide-presenting MHC molecule and/or the peptide fragment bound therein is to be carried out using MALDI methods. In MALDI analysis, the salt content of the matrix is frequently a critical parameter, since addition of ions may suppress the ionization or result in peak broadening or even interfering peaks. Using nanoparticles having a high ion exchange capacity to fix interfering salts in the matrix, this problem can be solved.

In a preferred embodiment of the process according to the invention for identifying and/or detecting T-cell epitopes, it is envisaged, that the second receptor unit, which is preferably present, is, prior to the competitive binding reaction, present free in solution, in particular in the case of MHC I molecules β-2-microglobulin. This means that, in this preferred embodiment, in which a second receptor unit is employed, the buffer used for carrying out the competitive binding reaction according to the invention comprises both the second receptor unit and the population of the peptide fragments of the protein antigen. The nanoparticles having the immobilized first receptor unit, the immobilization being effected by binding of the first functional group of the first receptor unit to the second functional group on the nanoparticle surface, are then added to the buffer which comprises the second receptor unit and the peptide fragment population and incubated in this buffer, where the first receptor unit, the second receptor unit and the at least one peptide fragment having affinity to both receptor units or the receptor or the receptor dimer formed by the two receptor units, may form a receptor/ligand complex, in particular a peptide-presenting MHC molecule. Here, the receptor/ligand complex formed is immobilized on the nanoparticles via the immobilized first receptor unit.

In a further preferred embodiment of the method according to the invention for identifying and/or detecting T-cell epitopes, it is envisaged that, prior to the competitive binding reaction, the second receptor unit together with the first receptor unit is immobilized on the nanoparticles in the form of a dimer which forms the receptor, in particular an MHC molecule. In this embodiment, it is envisaged that the second receptor unit has at least one third functional group and the surface of the nanoparticle has at least one complementary fourth functional group which binds the third functional group. Preferably, both receptor units which form the receptor dimer are immobilized on the nanoparticle in a targeted manner, where the biological activity of the receptor is maintained.

In the context of the present invention, the term “immobilized in a targeted manner” or “targeted immobilization” means that a molecule, in particular the receptor dimer, is immobilized on a nanoparticle in defined positions within the two receptor units in such a manner that the three dimensional structure of the domain(s) of the receptor required for biological activity is unchanged compared to the non-immobilized state and that this/these receptor domain(s), in particular the binding pocket for binding a suitable peptide, is/are, on contact with suitable peptides, freely accessible to these. “Immobilized in a targeted manner” also means that the two receptor units which form the receptor dimer are fixed in such a manner that the immobilized receptor, when used later on in a cellular or cell-like environment, cannot or only very slowly be degraded by protein-degrading enzymes. This means that the immobilized receptor dimer on the surface of the nanoparticles is arranged in such a manner that it offers the smallest possible number of points of attack for proteases.

“Maintaining the biological activity” means that the receptor units which form the receptor may, after immobilization on the surface of a nanoparticle, exert the same or almost the same biological functions to an extent which is at least similar to that of the same receptor units or the receptor formed by the two units in the non-immobilized state under suitable in vitro conditions, or the same receptor units or the same receptor in their/its natural cellular environment.

In the context of the present invention, a “dimer” or “receptor dimer” is to be understood as meaning a compound formed by the linkage of two subunits or units. The two linked receptor subunits are different molecules which may differ both in their composition, that is amino acid sequence, and with respect to their length. Preferably, in the immobilized receptor dimer, each receptor subunit or receptor unit is attached to the surface of the nanoparticle. According to the invention, it is also envisaged that only one receptor unit of the receptor dimer is fixed on the nanoparticle via a covalent bond between the first functional group and the second functional group.

According to the invention, it is envisaged that the third functional group of the second receptor unit is different from the first functional group of the first receptor unit and selected from the group consisting of carboxyl groups, amino groups, thiol groups, biotin groups, His tag, FLAG tag, Strep tag I groups, Strep tag II groups, histidine tag groups and FLAG tag groups.

According to the invention, it is envisaged that the third functional group is a natural component of the second receptor unit or is introduced into the second receptor unit by genetic engineering, enzymatic methods and/or chemical derivatization.

According to the invention, it is envisaged that the fourth functional group on the nanoparticle surface is different from the second functional group of the nanoparticles which binds the first functional group. The fourth functional group, i.e. the functional group on the surface of the nanoparticle, is, according to the invention, selected from the group consisting of amino groups, carboxyl groups, maleinimido groups, avidin groups, streptavidin groups, neutravidin groups and metal chelate complexes. According to the invention, it is envisaged that the fourth functional group, like the second functional group, is applied to the nanoparticle surface by graft silanization, silanization, chemical derivatization or similar suitable methods.

In a preferred embodiment of the invention it is envisaged that the first and second receptor unit are molecules which are naturally occurring or were prepared by genetic engineering or chemical synthesis, in particular chains of an MHC molecule.

In a preferred embodiment of the invention, the receptor is an MHC molecule of class I. According to the invention, preferably, the first receptor unit is a heavy chain of about 45 kDa and the second receptor unit is a light chain of about 12 dKa or the first receptor unit is a light chain of about 12 kDa and the second receptor unit is a heavy chain of about 45 kDa. It is, of course, also possible to employ modifications, mutations or variants of these chains, for example shortened forms of these chains, for example those where the transmembrane region is missing. Such truncated forms can, for example, be heavy chains without transmembrane region, having a molecular weight of 35 kDa. Thus, if, according to the invention, the first and the second receptor unit are capable of forming an MHC complex of class I, they can bind in the competitive binding reaction to a peptide fragment of about 8 to 18, preferably about 8 to 10, amino acids, thus forming a peptide-presenting receptor. Preferably, the heavy chain is an HLA-A, HLA-B or HLA-C monomer and the light chain is β-2-microglobulin.

In a further preferred embodiment of the invention, the receptor is an MHC molecule of class II. According to the invention, preferably, the first receptor unit is an α-chain of about 34 kD and the second receptor unit is a β-chain of about 30 kD or the first receptor unit is a β-chain of about 30 kD and the second receptor unit is an α-chain of about 34 kD. The α-chain and the β-chain are preferably HLA-DR, HLA-DQ or HLA-DP monomers. According to the invention, it also possible to use mutations, modifications or variants thereof. According to the invention, it is envisaged that, when the α-chain and the β-chain are used, the peptide fragment to be analyzed originates from an exogenous protein antigen. Thus, if, according to the invention, the first and the second receptor unit form an MHC complex of class II, they may bind a peptide fragment of about 8 to 18, preferably about 8 to 10, amino acids in the competitive binding reaction, thus forming a peptide-presenting receptor. According to the invention, it is envisaged that the first and the second receptor unit are natural chains or chains prepared by genetic engineering or chemical synthesis.

According to the invention, it is envisaged that the population of peptide fragments of the protein antigen to be analyzed is prepared by enzymatic protein cleavage, genetic engineering or chemical synthesis.

In a first embodiment of the invention, it is envisaged that the peptide fragments obtained in this manner of the prepared population completely represent the entire amino acid sequence of the protein antigen. In a second embodiment of the invention, it is envisaged that the peptide fragments of the population only partially represent the amino acid sequence of the protein antigen. These are in particular peptide fragments which, as determined using a computer algorithm, are potential T-cell epitopes. According to the invention, to predict potential T-cell epitopes, it is possible to employ computer algorithms such as SYFPEETHI (Rammensee et al., 1999) and HLA_BIND (Parker et al., 1994). If the receptor is an MHC molecule of type I, it is envisaged that the peptide fragments of the population to be produced have a length of 8 to 10 amino acids. If, in contrast, the receptor is an MHC molecule of type II, the peptide fragments of the population to be produced preferably have a length of 15 to 24 amino acids.

According to the invention, it is envisaged that the peptide fragments of the population can be provided with a marker and/or a fifth functional group. The marker serves in particular for detecting the peptide fragments. The marker can, for example, be a fluorescent marker or a radioactive marker. The fifth functional group of the peptide fragments serves preferably for the isolation and/or purification of the peptide fragments. For example, the peptide fragment bound in the peptide-presenting MHC molecule can, after release from the complex, be immobilized on suitable nanoparticles by binding of the fifth functional group to complementary sixth functional groups, and thus be removed from the other components of the complex. The fifth functional group is preferably different from the first, second, third and/or fourth functional group and is not able to form a bond with these.

In a preferred embodiment of the invention, the immobilization of the first receptor unit or the immobilization of the first and second receptor unit on the nanoparticles is effected by incubating the receptor unit(s) with the nanoparticles in a PBS buffer for a period of from one hour to four hours, preferably two hours, at room temperature in a shaking apparatus, giving nanoparticles having immobilized first receptor units or nanoparticles having immobilized first and second receptor units.

In a further embodiment of the invention, immobilization of receptor units on the nanoparticles can also be effected by preparing a peptide-presenting receptor using a peptide of known sequence and suitable length which is known to be capable of binding to the receptor used, i.e. the MHC molecule used, and the first receptor unit and the second receptor unit in solution. The peptide-presenting receptor prepared in this manner is then immobilized on the nanoparticles, and the nanoparticles obtained in this manner and having the immobilized peptide-presenting receptor are then subjected to a treatment to remove at least the bound peptide, giving nanoparticles having one or more immobilized receptor units. According to the invention, it is envisaged, in particular, that the peptide-presenting receptor is prepared by incubation of the first receptor unit, the second receptor unit and the peptide used in a buffer comprising 100 mM Tris, 2 mM EDTA, 400 mM L-arginine, 5 mM reduced glutathione and 0.5 mM oxidized glutathione for a period of more than 36 hours, preferably 48 hours, at a temperature of below 20° C., preferably 10° C.

If a first receptor unit having first functional groups and a second receptor unit having no functional third groups are used for preparing the peptide-presenting receptor, the peptide-presenting receptor prepared in solution is only immobilized on the nanoparticles by binding of the first functional group of the first receptor unit to the second functional group of the nanoparticles. If, in contrast, a first receptor unit having first functional groups and a second receptor unit having third functional groups are used to prepare the peptide-presenting receptor in solution, the receptor/ligand complex is immobilized on the nanoparticles by the bond between the first and the second functional group and the bond between the third and the fourth functional group.

Following immobilization of the peptide-presenting receptor on the nanoparticles, the nanoparticles obtained in this manner, having the immobilized receptor/ligand complex are treated with a stripping buffer, pH 3.0, which comprises 50 mM sodium citrate, for a period of less than 20 seconds, preferably 10 seconds. According to the invention, if the receptor/ligand complex is immobilized only by binding of the first functional group to the second functional group, in the treatment of the nanoparticles obtained in addition to the bound peptide the second receptor unit, too, is removed from the nanoparticles, giving a nanoparticle having the immobilized first receptor unit. If the peptide-presenting receptor is immobilized on the nanoparticles by binding of the first functional group of the first receptor unit to the second functional group of the nanoparticles and binding of the third functional group of the second receptor unit to the fourth functional group of the nanoparticles, in the treatment of the nanoparticles obtained with the stripping buffer, only the bound peptide is removed from the nanoparticles. Accordingly, this affords nanoparticles having the immobilized first and second receptor units. The nanoparticles prepared in this manner, which comprise either the immobilized first receptor unit or the immobilized first and second receptor unit can then, if appropriate after purification, be removed from the buffer, for example by at least one centrifugation and at least one washing, and be resuspended in a suitable buffer. The nanoparticles obtained in this manner can be used for carrying out the competitive binding reactions of the prepared population of peptide fragments.

According to the invention, it is envisaged that the competitive binding of the prepared peptide fragment population to the nanoparticles having the first or the first and second immobilized receptor unit is carried out by incubation of the peptide fragment population with the nanoparticles in a PBS buffer for a period of from 2 hours to 6 hours, preferably 4 hours, at a temperature of from room temperature to 39° C., preferably 37° C. If the nanoparticles have only the immobilized first receptor unit, the PBS buffer used for competitive binding also comprises the second receptor unit.

Following binding of the peptide fragment(s) having affinity to one or both receptor units, an immobilized receptor/ligand complex is obtained which is then, by centrifugation and at least one washing, removed from the buffer and the unbound peptide fragments of the population and resuspended in a buffer.

According to the invention, the analysis of the resulting peptide-presenting receptor and/or the bound peptide fragment is then carried out. According to the invention, it is envisaged that the suspension of the nanoparticles having the immobilized peptide-presenting receptor with the bound peptide are analyzed by matrix-assisted laser desorption ionization (MALDI) methods.

MALDI methods are mass spectrometry methods. Mass spectrometry is a method for elucidating the structure of substances where atomic and molecular particles are separated according to their mass. It is based on a reaction between molecules and electrons or photons. By bombarding the sample with electrons, as a result of the splitting-off of electrons, positive molecular ions are formed which then disintegrate into various ionic, radical and/or neutral fragments. Molecular ions and fragments are separated in suitable separation systems according to their mass number. Thus, in mass spectrometry, the fragments or molecular ions formed by chemical disintegration reactions as a result of an ionization process are used for elucidating the structure of substances. A MALDI method which is preferred according to the invention is the MALDI-TOF MS method (matrix-assisted laser desorption ionization time-of-flight mass spectrometry). The main advantages of this method are the extremely quick positive identification of the substance to be analyzed, for example a protein or peptide, by its mass/charge ratio (m/z), and the extremely low detection threshold, which is in the femtomol range or below.

According to the invention, it is envisaged, in particular, to deposit and analyze the nanoparticles obtained, for example, in the form of a suspension, after centrifugation and washing on a MALDI sample stage or MALDI target. Here, a matrix employed during the MALDI method, in particular MALDI-TOF MS method, can be applied before or after the deposition of the nanoparticle-containing suspension or jointly therewith on the MALDI sample stage.

In a further embodiment of the invention, it is envisaged that the at least one peptide fragment bound in the immobilized receptor/ligand complex is removed from the receptor by dissolution, isolated and analyzed. To release the peptide fragment, the nanoparticles having the immobilized receptor with the at least one bound peptide fragment can be treated, for example, in a stripping buffer, pH 3.0, comprising 50 mM sodium citrate, for a period of less than 20 seconds, preferably 10 seconds. According to the invention, it is also possible to isolate and purify the at least one peptide fragment using nanoparticles, if the fragment has a fifth functional group. In this case, these nanoparticles contain a sixth functional group which binds the fifth functional group, so that it is possible to isolate specifically the released peptide fragments from an aqueous solution or suspension. According to the invention, it is envisaged that the at least one isolated peptide fragment is then sequenced.

The present invention also relates to a method for preparing a peptide vaccine against a protein antigen, in particular against cells or biological materials expressing or presenting the protein antigen, where the amino acid sequence of a T-cell epitope of the protein antigen is identified in vitro, a peptide having the identified amino acid sequence is prepared and, in a preferred embodiment, a receptor/ligand complex, in particular a peptide-presenting MHC molecule, which can be used as vaccine, is then prepared using the prepared peptide and a first and, if appropriate, second receptor unit, in particular a first and second chain of an MHC molecule. The method according to the invention comprises
a) providing a population of peptide fragments of the protein antigen,
b) providing nanoparticles having, at their surface, at least one first immobilized chain of an MHC molecule, where the chain has a conformation which allows formation of an MHC molecule,
c) carrying out competitive binding of the peptide fragment population to the first chain immobilized on the nanoparticles optionally and preferably in the presence of a second chain of an MHC molecule, where the peptide fragment having affinity, in particular having the greatest affinity to the first chain, in particular to the two chains of the MHC molecule, binds if appropriate together with the second chain to the first chain, giving a peptide-presenting MHC molecule, and
d) isolation of the peptide fragment from the MHC molecule and determination of its amino acid sequence to obtain the peptide vaccine which can be used in the form of the peptide fragment itself or of its MHC complex.

This may optionally be followed by the following steps:
1) preparation, by genetic engineering or chemical synthesis, of suitable amounts of a peptide based on the determined amino acid sequence of the peptide fragment,
2) preparation, by genetic engineering or chemical synthesis, of suitable amounts of the first and second chains,
3) preparation of suitable amounts of peptide-presenting MHC molecules by joint incubation of the first chain, the second chain and the peptide prepared, and
4) preparation of a peptide vaccine in the form of a lyophilizate or an aqueous colloidal solution or suspension of the peptide-presenting MHC molecules.

In the context of the present invention, a “vaccine” is to be understood as meaning a composition for generating immunity for the prophylaxis and/or treatment of diseases. Accordingly, vaccines are medicaments which comprise antigens and are intended to be used in humans or animals for generating specific defense and protective substance by vaccination. Vaccines are used for the active formation of antibodies.

Here, it is envisaged according to the invention to produce the population of peptide fragments of the protein antigen by enzymatic protein cleavage, genetic engineering or chemical synthesis. In a preferred embodiment, the peptides present in the peptide population completely represent the entire amino acid sequence of the protein antigen. In an alternative embodiment, the peptide fragments present in the peptide population only partially represent the amino acid sequence of the protein antigen, the peptide fragments of the population preferably having amino acid sequences which represent potential T-cell epitopes determined using a computer algorithm. According to the invention, it is envisaged that the peptide fragments have a length of 8 to 10 amino acids if the MHC molecule to be prepared is an MHC molecule type I. If the MHC molecule to be prepared is an MHC molecule type II, the peptide fragments preferably have a length of 15 to 24 amino acids.

If the MHC molecule to be prepared is an MHC molecule type I, the first chain is a heavy chain of about 45 kDa and the second chain is a light chain of about 12 kDa. In this case, the first chain is in particular an HLA-A, HLA-B or HLA-C monomer and the second chain is β-2-microglobulin.

If the MHC molecule to be prepared is an MHC molecule type II, according to the invention, the first chain is an α-chain of about 34 kDa and the second chain is a β-chain of about 30 kDa. In this case, the first chain and the second chain are preferably HLA-DR, HLA-DQ or HLA-DP monomers. Both the chains of the MHC type I and the MHC type II classes can be employed in mutated, changed or modified, in particular shortened, form.

Preferably, the first chain contains a first functional group, so that the first chain is immobilized on the surface of the nanoparticles by binding of the first functional group to a second functional group present on the surface of the nanoparticles. According to the invention, it is envisaged that the functional group is a natural component of the first chain or is introduced into the first chain by genetic engineering, biochemical, enzymatic and/or chemical derivatization or chemical synthesis. The first functional group is preferably a group selected from the group consisting of carboxyl groups, amino groups, thiol groups, biotin groups, His tag, FLAG tag, Strep tag I groups, Strep tag II groups, histidine tag groups and FLAG tag groups. The second functional group present on the surface of the nanoparticles is preferably selected from the group consisting of amino groups, carboxyl groups, maleinimido groups, avidin groups, streptavidin groups, neutravidin groups and metal chelate complexes. Here, the second functional group can be applied to the surface of the nanoparticles by graft silanization, silanization, chemical derivatization and similar suitable processes. The nanoparticles to be used are preferably those having a core of a chemically inert material, preferably silicon dioxide, and a diameter of from 30 to 400 nm, preferably from 50 nm to 150 nm.

In a preferred embodiment, the nanoparticles which have a first immobilized chain on their surface are obtained by the following steps:
a) incubation of the first chain which contains the first functional group, of the second chain and of a peptide whose amino acid sequence is known and which is known to be capable of forming an MHC molecule under suitable conditions,
b) incubation of the MHC molecule formed with nanoparticles whose surface has a second functional group which binds the first functional group, under conditions suitable for immobilizing the MHC molecule on the nanoparticles,
c) treatment of the nanoparticle having the immobilized MHC molecules with a suitable buffer to remove the second chain and the peptide having a known amino acid sequence from the MHC molecule, and
d) purification of the nanoparticles having the first immobilized chain.

According to the invention, it is preferably envisaged that the competitive binding of the peptide fragment population to the nanoparticles having the first immobilized chain is carried out by incubating the peptide fragment population with the nanoparticles in a suitable buffer under suitable conditions. After binding of the at least one peptide fragment having affinity of the population and, if appropriate, the second chain with formation of an immobilized MHC molecule, the nanoparticles having the immobilized MHC molecule are removed by centrifugation and washing from the buffer and the unbound peptide fragments. The nanoparticles having the immobilized MHC molecule are then treated with a buffer, for example a stripping buffer, suitable for releasing the bound peptide fragment. The released peptide fragment is then isolated and its amino acid sequence is determined.

Based on the determined amino acid sequence, the bound peptide fragment can then be prepared in large amounts using, for example, genetic engineering. Based on the determined amino acid sequence of the released peptide fragment, it is possible, for example, to generate a nucleic acid coding for the determined amino acid sequence and insert it into a suitable expression vector. This vector is then transferred into a host cell suitable for expressing the amino acid sequence. In this manner, it is possible to express relatively large amounts of a peptide in the host cell and to isolate it therefrom.

Based on the determined amino acid sequence of the released peptide fragment, it is also possible to prepare a relatively large amount of peptide synthetically.

The present invention also relates to a method for controlling the quality of receptor/ligand complexes and/or components thereof, which comprises preparing or providing a receptor/ligand complex in solution of at least one receptor unit, preferably two receptor units, where at least one receptor unit has a first functional group, and a ligand, immobilizing the receptor/ligand complexes on nanoparticles which have, on their surface, at least one second functional group which binds the first functional group, and analyzing the nanoparticles having the immobilized receptor/ligand complex using a MALDI method.

Preferably, the receptor is an MHC molecule, the ligand is a peptide of known sequence and defined length which binds to the receptor and the receptor/ligand complex is a peptide-presenting MHC molecule.

In one embodiment, the receptor is an MHC molecule of class I, where one receptor unit is a heavy chain of about 45 kDa and one receptor unit is a light chain of about 12 kDa. Here, the heavy chain is an HLA-A, HLA-B or HLA-C monomer and the light chain is β-2-microglobulin.

In a further embodiment of the method according to the invention, the receptor is an MHC molecule of class II, where one receptor unit is an α-chain of about 34 kDa and one receptor unit is a β-chain of about 30 kDa. Here, the α-chain and the β-chain are HLA-DR, HLA-DQ or HLA-DP monomers.

According to the invention, for the analysis, a MALDI method, in particular a MALDI-TOF method, is used.

The present invention also relates to a method for preparing nanoparticles having, on their surface, at least one immobilized receptor unit or one immobilized receptor, which comprises
a) preparing a receptor/ligand complex by incubation of a first receptor unit having a first functional group, if appropriate, in a preferred embodiment of a second receptor unit capable of forming, with the first receptor unit, a receptor, and a ligand in solution,
b) immobilizing the receptor/ligand complex formed on nanoparticles having, on the surface, at least one second functional group which binds the first functional group, and
c) treating the nanoparticles having the immobilized receptor/ligand complex with an acidic buffer to release at least the bound ligand, giving nanoparticles having immobilized receptor units.

In one embodiment of the invention, the immobilization of the receptor/ligand complex on the nanoparticle surface is only carried out via the first functional group of the first receptor unit binding to the second functional group of the nanoparticles. In this case, after the treatment, with an acidic buffer, of the nanoparticles having the immobilized receptor/ligand complex in addition to the ligand the second receptor unit, too, is released, giving nanoparticles having the immobilized first receptor unit.

In a further embodiment of the method according to the invention, the second receptor unit has a third functional group, while the nanoparticles have, on their surface, a fourth functional group which binds the third functional group of the second receptor unit. Thus, the receptor/ligand complex on the nanoparticles is immobilized via binding of the first functional group of the first receptor unit to the second functional group of the nanoparticles and binding of the third functional group of the second receptor unit to the fourth functional group of the nanoparticles. In this case, after the treatment of the nanoparticles having the immobilized receptor/ligand complex with an acidic buffer only the ligand is released, giving nanoparticles having immobilized first and second receptor units. Preferably, the first and second receptor units are immobilized in a targeted manner, forming a receptor capable of binding a ligand.

In a preferred embodiment, the receptor is an MHC molecule, the ligand is a peptide of known sequence and defined length which binds to the receptor and the receptor/ligand complex is a peptide-presenting MHC molecule.

In particular, the receptor is an MHC molecule of class I which, as first unit, has a heavy chain of about 45 kDa and, as second receptor unit, has a light chain of about 12 kDa or, as first receptor unit, has a light chain of about 12 kDa and, as second receptor unit, has a heavy chain of about 45 kDa. The heavy chain is an HLA-A, HLA-B or HLA-C monomer and the light chain is β-2-microglobulin.

In a further preferred embodiment of the invention, the receptor is an MHC molecule of class II which, as first receptor unit, has an α-chain of about 34 kDa and, as second receptor unit, has a β-chain of about 30 kDa or, as first receptor unit, has a β-chain of about 30 kDa and, as second receptor unit, has an α-chain of about 34 kDa. The α-chain and the β-chain are HLA-DR, HLA-DQ or HLA-DP monomers.

According to the invention, it is envisaged that the first functional group and the third functional group are different from one another and are selected from the group consisting of carboxyl groups, amino groups, thiol groups, biotin groups, His tag, FLAG tag, Strep tag I groups, Strep tag II groups, histidine tag groups and FLAG tag groups.

According to the invention, it is also envisaged that the second functional group on the surface of the nanoparticle, which binds the first functional group, and the fourth functional group on the surface of the nanoparticle, which binds the third functional group, are different from one another and selected from the group consisting of amino groups, carboxyl groups, maleinimido groups, avidin groups, streptavidin groups, neutravidin groups and metal chelate complexes.

Preferably, the nanoparticles which have the immobilized receptor/peptide complex are treated with a stripping buffer, pH 3.0, which comprises 50 mM sodium citrate, for a period of less than 20 s, preferably 10 s, to remove the bound peptide.

The present invention also relates to a method for preparing nanoparticles having immobilized peptide-presenting MHC molecules, where nanoparticles having at least one first immobilized chain of an MHC molecule which were prepared by a method according to the invention for preparing nanoparticles having at least one immobilized receptor unit or having an immobilized receptor, are incubated in the presence of a second chain capable of forming an MHC molecule with the first chain, with a peptide capable of binding to the MHC molecule, giving a peptide-presenting MHC molecule immobilized on the nanoparticles.

The MHC molecule is preferably a molecule of class I, where the peptide has a length of about 8 to about 10 amino acids. The MHC molecule may also be a molecule of class II where the peptide has a length of about 15 to about 24 amino acids.

The present invention also relates to a method for enriching and/or isolating specific CD4⁺-T-lymphocytes or CD8⁺-T-lymphocytes from peripheral blood mononuclear cells (PBMCs), which comprises
a) preparing nanoparticles having immobilized peptide-presenting MHC molecules, where the peptide is a T-cell epitope,
b) isolating peripheral blood mononuclear cells from a suitable starting material,
c) incubating the isolated blood mononuclear cells with the nanoparticles having the immobilized peptide-presenting MHC molecules, the T-lymphocytes binding to the T-cell epitope of the immobilized peptide-presenting MHC molecules,
d) removing the nanoparticles having the T-lymphocytes bound to the immobilized peptide-presenting MHC molecules from the unbound peripheral mononuclear cells.

According to the invention, it is envisaged that the bound T-lymphocytes are then released from the nanoparticles and, in vitro, propagated clonally. The released and/or clonally propagated T-lymphocytes can then, for example, be introduced into an organism.

In a preferred embodiment of the invention, the peptide-presenting MHC molecule is a molecule of class I and the bound T-lymphocytes are CD8⁺-T-lymphocytes. In a further preferred embodiment, the peptide-presenting MHC molecule is a molecule of class II, the bound T-lymphocytes being CD4⁺-T-lymphocytes.

The present invention also relates to a method for priming and/or restimulating a CD4⁺-T- or CD8⁺-T-lymphocyte reaction in vitro, which comprises
a) identifying a T-cell epitope and determining its amino acid sequence,
b) preparing a nucleic acid coding for a peptide having the amino acid sequence of the T-cell epitope,
c) introducing the nucleic acid prepared under b) into a suitable vector,
d) introducing the vector obtained under c) into dendritic cells isolated, if appropriate, from cultivated peripheral blood mononuclear cells,
e) propagating the dendritic cells obtained under d), which have the vector, in vitro, and
f) stimulating autologous CD4⁺- and/or CD8⁺-cells in vitro using the dendritic cells obtained under d) or e).

The present invention also relates to nanoparticles comprising on the surface at least one receptor unit, in particular an immobilized chain of an MHC molecule. Here, the immobilized chain may, by binding a peptide of 8 to 24 amino acids and a second chain of an MHC molecule, form a peptide-presenting MHC molecule. The MHC molecule chain is immobilized on the nanoparticle surface by binding of a first functional group present in the chain to a second functional group present on the nanoparticle surface. In the nanoparticles according to the invention, either the heavy chain or the light chain of an MHC molecule of class I or either the α-chain or the β-chain of an MHC molecule of class II is in immobilized form.

The present invention also relates to nanoparticles having an immobilized MHC molecule, where the MHC molecule comprises a first and a second chain and the MHC molecule is immobilized on the nanoparticle surface by binding of a first functional group present in the first chain to a second functional group present on the nanoparticle surface or by binding of the first functional group present in the first chain to the second functional group present on the nanoparticle surface and binding of a third functional group present in the second chain to a fourth functional group present on the nanoparticle surface.

The present invention likewise relates to nanoparticles having a peptide-presenting MHC molecule immobilized on the nanoparticle surface, where the peptide-presenting MHC molecule comprises a first chain, a second chain and a peptide of 8 to 24 amino acids and the MHC molecule is immobilized on the nanoparticle surface by binding of a first functional group present in the first chain to a second functional group present on the nanoparticle surface or by binding of the first functional group present in the first chain to the second functional group present on the nanoparticle surface and binding of a third functional group present in the second chain to a fourth functional group present on the nanoparticle surface.

Furthermore, the present invention relates to a peptide vaccine which comprises at least one peptide-presenting MHC molecule or the peptide fragment identified according to the invention itself, the peptide vaccine being obtainable by the method according to the invention.

In one embodiment, the peptide vaccine may be present as a lyophilizate. In another embodiment, the vaccine is present as an aqueous colloidal solution or suspension. Additionally, the peptide vaccine according to the invention may comprise at least one adjuvant.

The present invention also relates to a kit for identifying and/or detecting T-cell epitopes of a protein antigen in vitro, comprising a container with a suspension of nanoparticles having an immobilized MHC molecule. In a further embodiment, the kit may comprise a container with a suspension of nanoparticles having first chains of an MHC molecule immobilized thereon and a container with a lyophilizate of a second chain.

The present invention also relates to the use of a nanoparticle according to the invention for identifying and/or detecting T-cell epitopes of a protein antigen in vitro.

Furthermore, the present invention relates to the use of a nanoparticle according to the invention for preparing a peptide vaccine.

Further, the present invention relates to the use of a nanoparticle for enriching and/or isolating specific CD4⁺-T-lymphocytes or CD8⁺-T-lymphocytes in vitro.

Furthermore, the present invention relates to the use of a nanoparticle according to the invention for priming and/or restimulating a CD4⁺-T- and/or CD8⁺-T-lymphocyte reaction in vitro. The present invention likewise relates to the use of a peptide vaccine according to the invention for the active immunization of an animal or human organism against a protein antigen.

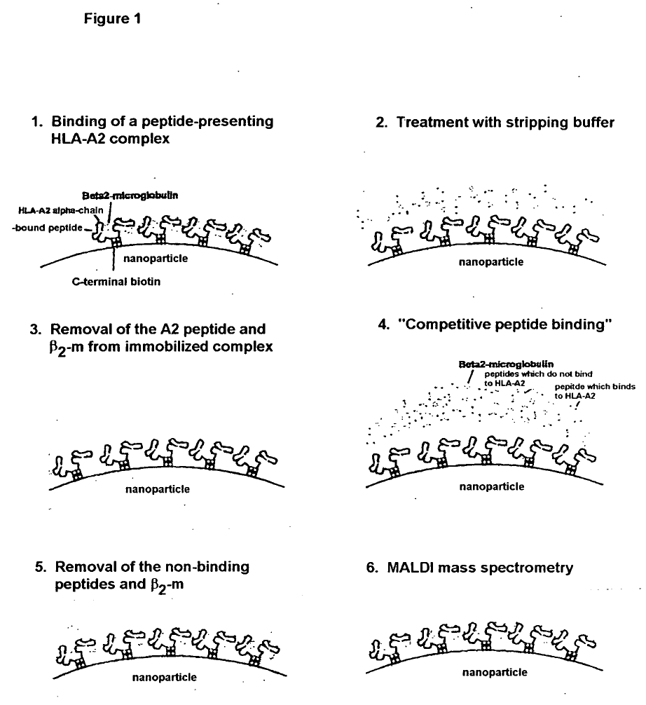

The present invention is illustrated in more detail by the FIGS. 1 to 3 and the examples below.

FIG. 1 shows, in schematic form, a preferred embodiment of the method according to the invention for identifying and/or detecting T-cell epitopes, where a peptide-presenting HLA-A2 complex prepared in solution is immobilized on nanoparticles. The nanoparticles having the complex are then treated with an acidic stripping buffer, resulting in the removal of the EBV-EBNA-6 peptide (positions 284-293, LLDFVRFMGV) and β-2-microglobulin (β₂-m). The nanoparticles prepared in this manner having the immobilized HLA chain are then used for carrying out a competitive binding reaction using a peptide population in the presence of β₂-m, where the peptide(s) having affinity binds/bind to HLA and β₂-m, resulting in the formation, on the nanoparticle surface, of an HLA complex presenting this/these peptide(s). Following removal of the unbound peptides and excess β₂-m, the nanoparticles having the immobilized peptide-presenting complex are subjected to analysis by MALDI mass spectrometry.

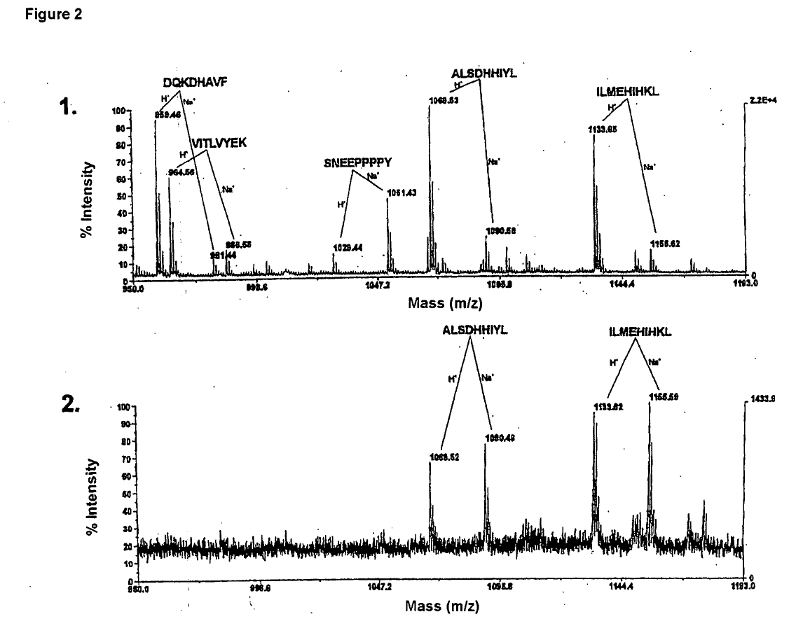

FIG. 2 shows mass spectrograms, obtained by MALDI mass spectrometry, of nanoparticles having immobilized peptide-presenting HLA complexes. FIG. 2.1 relates to the peptide mixture of equimolar amounts of the 5 peptides mentioned in Example 4 and FIG. 2.2 relates to the two peptides identified after selection as binding.

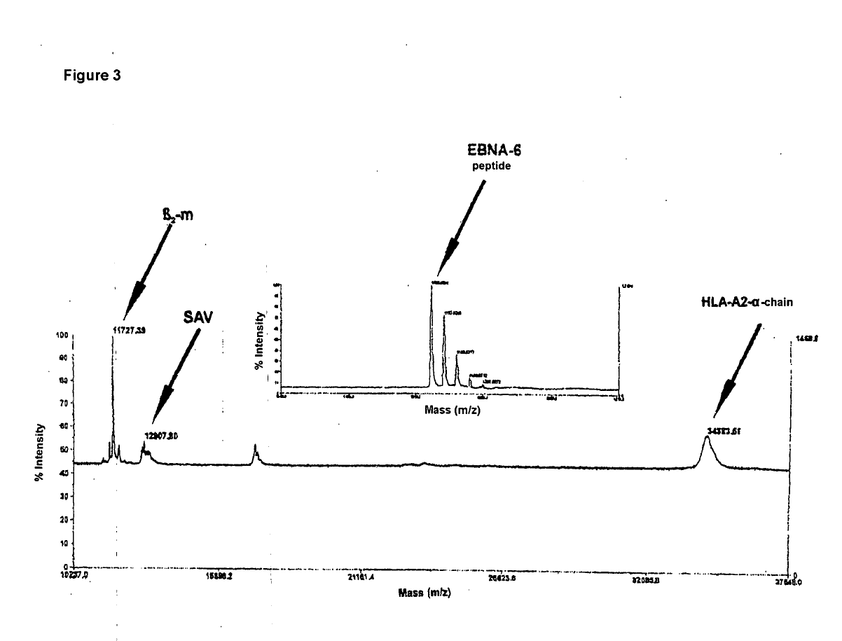

FIG. 3 shows the MALDI spectrum of all molecular components of the HLA-A2-EBNA-6 complex immobilized on SAV nanoparticles. The insert shows the MALDI spectrum of the EBNA-6 peptide [M+H]⁺ having the sequence LLDFVRFMGV (theoretical monoisotropic mass [M+H]⁺ 1196.6502 μ). The peak at 11727 denotes β₂-m, the peaks at about 12900 denote the SAV-nanoparticles in monomeric form and the peak at 34383 denotes the biotinylated alpha chain.

### EXAMPLE 1

### Peptide Synthesis

Peptides were synthesized using the Fmoc solid phase method on a continuous MillGen 9050 flow synthesis apparatus (Millipore, Bedford, USA). After purification by RP-HPLC, the peptides were lyophilized and dissolved in PBS buffer at a concentration of 1 mg/ml.

### EXAMPLE 2

### Preparation of Soluble Biotinylated HLA-A2 Monomers

Soluble HLA-A*0201 peptide tetramers were synthesized as described by Altman et al., Science, 274 (1996), 94-96. Recombinant heavy HLA-A*0201 chains (positions 1-276) in soluble form and β-2-microglobulin (β₂-m) were expressed separately in Escherichia coli cells which had been transformed using appropriate expression plasmids. The 3′-terminus of the extracellular domains of the heavy HLA-A*0201 chain were modified using a BirA biotinylation sequence. The Escherichia coli cells which had been transformed with the appropriate expression plasmids coding for the HLA-A*0201 chain or β₂-m were cultivated until they reached the mid-log growth phase. They were then induced using 0.5 isopropyl β-galactosidase. After further cultivation and expression of the recombinant proteins, the Escherichia coli cells were harvested and purified. After cell disruption, the inclusion bodies present in the cells were isolated, purified and solubilized in 8 M urea, pH 8.0. The heavy HLA-A*0201 chain and β₂-m were diluted in 100 mM Tris, 2 mM EDTA, 400 mM L-arginine, 5 mM reduced glutathione and 0.5 mM oxidized glutathione, and 10 μM of the peptide LLDFVRFMGV (EBV EBNA-6, positions 284-293) were added. The mixture was then incubated with stirring at 10° C. for 48 hours. The folded 48 kDa complexes (α-chain: about 35 kDa, β₂-m: about 12 kDa, peptide: about 1 kDa) were concentrated by ultrafiltration using a membrane having a retention capacity of 10 kDa (Millipore, Bedford, USA) and purified by HPSEC using a Superdex G75 HiLoad 26/60 column (Amersham Pharmacia Biotech, Upsala, Sweden) and 150 mM NaCl, 20 mM Tris-HCl, pH 7.8, as elution buffer. Following gel filtration, the purified monomers were biotinylated using a biotin ligase (BirA; Avidity, Denver, USA) and repurified by HPSEC. The complex was then adjusted to a concentration of 1 μg/μl by ultrafiltration.

### EXAMPLE 3

### Preparation and Characterization of Streptavidin-Modified Nanoparticles (SAV-Nanobeads)

Silicon dioxide particles were prepared as described by Stoeber et al., J. Coll. inter. Sci., 26 (1968), 62-62. Spherical silicon dioxide particles having a mean hydrodynamic particle diameter of 100 nm were obtained, as determined by dynamic light scattering measurements using a Zetasiser 3000 HSA apparatus (Malvern Instruments, Herrenberg, Germany). 500 μg of the carboxy-modified particles were mixed with 15 μg of streptavidin (Roche, Tutzing, Germany). The immobilized streptavidin was quantified by quenching the fluorescence of biotin-4-fluorescein. It was found that the entire 15 μg of streptavidin were immobilized on the nanoparticles. About 57% of the theoretical biotin binding sites were freely accessible on the particle surface. Since d_{silicon dioxide}=100 nM, D_{silicon dioxide}=4 g/ml and M_{streptavidin}=52 kDa, about 730 streptavidin tetramers were bound on each particle, so that about 1600 biotin binding sites were freely accessible on the surface. The streptavidin-modified particles were adjusted to a concentration of 0.5 mg/ml in PBS.

### EXAMPLE 4

### HLA-A2 Peptide Selection Test

All washing steps of the nanoparticles were carried out by centrifuging for 10 minutes at 15 000×g at 20° C. in a temperature-controlled centrifuge in 1.5 ml reaction vessels and by resuspending the beads using a micropipette. 55 μg of SAV nanoparticles and 3.5 μg of the soluble HLA-A2 complex comprising the peptide LLDFVRFMGV (EBV EBNA-6, positions 284-293) were suspended in 20 μl of PBS. For 2 hours, the mixture was incubated in a horizontal shaker at room temperature to prevent sedimentation. After 10 minutes of centrifugation at 20° C., the supernatant was discarded and the nanoparticles were washed with 50 μl of water. To release β₂-m molecules and the peptide LLDFVRFMGV comprised in the complex, the beads were incubated for 90 seconds in 150 μl of stripping buffer (50 mM sodium citrate, pH 3.0) and, after centrifugation, washed with 150 μl of water. The beads were then resuspended using 30 μl of PBS containing 1.2 μg of β₂-m molecules (Sigma, Munich, Germany) and a peptide mixture. The mixture consisted of a total of 5 peptides in an amount of in each case 0.072 μg. The 5 peptides had the sequences ILMEHIHKL, DQKDHAVF, ALSDHHIYL, VITLVYEK and SNEEPPPPY. After four hours of incubation at 37° C., the nanoparticles were pelleted by centrifugation and, after removal of the supernatant, washed with 50 μl of PBS buffer and then 50 μl of water. After the final centrifugation, the nanoparticles were resuspended in 0.1% water/TFA (v/v) and transferred to a MALDI target. Analysis was carried out using a Voyager DE-STR mass spectrometer (Applied Biosystems Foster City, USA) in positive ion reflection mode. Solutions comprising proteins and peptides were mixed on the target with an identical matrix volume using a 1:20 dilution of saturated α-cyano-4-hydroxycinnamic acid or sinapinic acid in 30% acetonitrile/0.3% TFA (v/v). All MALDI spectra were calibrated externally using a standard peptide mixture.

According to the invention, the following results were obtained:

All components of the biotinylated HLA-A2 complex can be detected and determined quantitatively using the MALDI-TOF method.

Complete complexes immobilized via biotin on the SAV particles (SAV nanobeads) were visualized by MALDI mass spectrometry, the corresponding mass signals for the biotinylated HLA-A2 α-chain being 34379 Da, that for β₂-m molecules being 11727 Da, that for the streptavidin monomer being 12907 Da and that for the bound peptide LLDFVRFMGV being 1196.63 Da (FIG. 3). Using the MALDI-TOF method, it was thus possible to check both the correct properties of the HLA-A2 complex on the one hand and the effectiveness of the method for immobilizing the biotinylated complex on the SAV nanoparticles.

Under conditions of competitive binding and using a peptide mixture, HLA-A2-complexed SAV nanoparticles bind only the peptides predicted for HLA-A2.

FIG. 2 shows the MALDI spectra of a peptide mixture comprising two HLA-A2 peptides which bind and three peptides which do not bind, each peptide being present in an amount of about 70 pmol. The predicted binding of the peptides was determined using the SYFPEITHI program, where, at very strong binding, a score of 32 was determined for the peptide ILMEHIHKL, at a very strong binding, a score of 23 was determined for the peptide ALSDHHIYL and for the three nonbinding proteins a score of 0 was determined. The different signal intensities of the respective peptides in the mixture used are the result of different ionization capacities. The identity of the observed peaks was confirmed by MALDI-PSD sequencing. Following selection of the peptides having HLA receptors, after treatment, i.e. washing with PBS buffer, only the signals for the binding peptides remained. The fact that no signal could be detected for the nonbinding proteins shows that there are no unspecific interactions. The spectra show the monoisotopic mass for each peptide in protonated form ([M+H]⁺) and the monoisotope in sodium form ([M+Na]⁺).

1. A method for at least one of identifying and detecting T-cell epitopes of a protein antigen in vitro, where a population of peptide fragments of the antigen is subjected to competitive binding to a first immobilized receptor unit, preferably in the presence of a second receptor unit which, together with the first receptor unit, is capable of forming a receptor, where at least one peptide fragment with affinity to the receptor binds to at least the first receptor unit(s), and the bound peptide fragment is then isolated and analyzed, said method comprising
a) immobilizing at least the first receptor unit which has at least one first functional group on a nanoparticle, the surface of which has at least one second functional group which binds the first functional group,
b) preparing of a population of peptide fragments of the protein antigen which comprises different sequence ranges of the protein antigen,
c) carrying out a competitive binding of the peptide fragment population to the first receptor unit immobilized on the nanoparticle where at least one peptide fragment having affinity to at least the first receptor unit binds to the first receptor unit, giving a receptor/peptide fragment complex immobilized on the nanoparticle, and
d) analyzing the at least one of immobilized receptor/peptide fragment complex and the bound peptide fragment.

2-5. (canceled)

6. The method as claimed in claim 1, where the receptor is a major histocompatibility complex (MHC) molecule, the receptor/peptide fragment complex is a peptide-presenting MHC molecule and the first and the second receptor unit are chains of the MHC molecule.

7-16. (canceled)

17. The method as claimed in claim 1, where the first and the second receptor unit are either natural chains or chains prepared by genetic engineering or chemical synthesis.

18-24. (canceled)

25. The method as claimed in claim 1, where the population of peptide fragments of the protein antigen is prepared by a method selected from the group consisting of enzymatic protein cleavage, genetic engineering and chemical synthesis.

26-34. (canceled)

35. The method as claimed in claim 1, where the immobilization of the first receptor unit or the immobilization of the first and second receptor unit on the nanoparticles is carried out by incubating the receptor unit(s) with the nanoparticles in a PBS buffer for a period of 1 h to 4 h at room temperature in a shaking apparatus, affording nanoparticles having immobilized first receptor units or nanoparticles having immobilized first and second receptor units.

36. The method as claimed in claim 1, where the immobilization of receptor unit(s) on the nanoparticles is carried out by preparing in solution a receptor/peptide complex using a peptide of known sequence and suitable length, the first receptor unit and the second receptor unit, immobilizing the receptor/peptide complex on the nanoparticles, subjecting the nanoparticles having the immobilized receptor/peptide complex to a treatment to remove at least the bound peptide, resulting in nanoparticles having immobilized receptor units.

37-46. (canceled)

47. The method as claimed in claim 1, where the suspension of the nanoparticles having the immobilized receptor/peptide fragment complex with the bound peptide fragment is analyzed using a matrix-assisted laser desorption/ionization (MALDI) method.

48-49. (canceled)

50. The method as claimed in claim 1, where the peptide fragment bound in the immobilized receptor/peptide fragment complex is removed from the complex by dissolution, isolated and analyzed.

51-54. (canceled)

55. A method for at least one of identifying and preparing a peptide vaccine against a protein antigen, where an amino acid sequence of a T-cell epitope of the protein antigen is identified in vitro, a peptide having the identified amino acid sequence is prepared and a peptide-presenting major histocompatibility complex (MHC) is prepared using the prepared peptide and a first and second chain, which method comprises:
a) providing a population of peptide fragments of the protein antigen,
b) providing nanoparticles having, at their surface, at least one first immobilized chain of an MHC molecule, where the chain has a conformation which allows formation of an MHC molecule,
c) carrying out competitive binding of the peptide fragment population to a first chain immobilized on the nanoparticles in the presence of a second chain of an MHC molecule, where a peptide fragment having the greatest affinity to the two chains of the MHC molecule binds together with the second chain to the first chain, giving a peptide fragment-presenting MHC molecule, and
d) isolating the peptide fragment from the MHC molecule to identify a peptide fragment suitable for a peptide vaccine, and determining its amino acid sequence.

56-74. (canceled)

75. The method as claimed in claim 55, where the nanoparticles which have a first immobilized chain on their surface are obtained by the following steps:
a) incubating the first chain which contains the first functional group, of the second chain and of a peptide whose amino acid sequence is known and which is known to be capable of forming a peptide-presenting MHC molecule under suitable conditions,
b) incubating of the peptide-presenting MHC molecule with nanoparticles whose surface has at least one second functional group which binds the first functional group, under conditions suitable for immobilizing the peptide-presenting MHC molecule on the nanoparticles,
c) treating the nanoparticles having the immobilized peptide-presenting MHC molecules with a suitable buffer to remove the second chain and the peptide having a known amino acid sequence from the immobilized MHC molecule, and
d) purifying of the nanoparticles having the first immobilized chain.

76-81. (canceled)

82. A method for controlling the quality of receptor/ligand complexes and/or components thereof, which comprises preparing or providing a receptor/ligand complex in solution of two receptor units, where at least one receptor unit has a first functional group, and a ligand, immobilizing the receptor/ligand complexes on nanoparticles which have, on their surface, at least one second functional group which binds the first functional group, and analyzing the nanoparticles having the immobilized receptor/ligand complex using a MALDI method.

83-88. (canceled)

89. A method for preparing nanoparticles having, on their surface, at least one immobilized receptor unit or one immobilized receptor, which comprises
a) preparing a receptor/ligand complex by incubation of a first receptor unit having a first functional group, a second receptor unit capable of forming, with the first receptor unit, a receptor, and a ligand in solution,
b) immobilizing the receptor/ligand complex formed on nanoparticles having, on the surface, at least one second functional group which binds the first functional group, and
c) treating the nanoparticles having the immobilized receptor/ligand complex with an acidic buffer to release at least the bound ligand, giving nanoparticles having immobilized receptor units.

90-94. (canceled)

95. The method as claimed in claim 89, where the receptor is an MHC molecule, the ligand is a peptide of known sequence and defined length which binds to the receptor and the receptor/ligand complex is a peptide-presenting MHC molecule.

96-104. (canceled)

105. A method for preparing nanoparticles having immobilized peptide-presenting MHC molecules, where nanoparticles having at least one first immobilized chain of an MHC molecule prepared by a method according to claim 89 are incubated in the presence of a second chain capable of forming an MHC molecule with the first chain, with a peptide capable of binding to the MHC molecule, giving a peptide-presenting MHC molecule immobilized on the nanoparticles.

106-107. (canceled)

108. A method for at least one of enriching and isolating specific CD4⁺-T-lymphocytes or CD8⁺-T-lymphocytes from peripheral blood mononuclear cells (PBMCs), which comprises
a) preparing nanoparticles having immobilized peptide-presenting MHC molecules as claimed in claim 105, where the peptide is a T-cell epitope,
b) isolating peripheral blood mononuclear cells from a suitable starting material,
c) incubating the isolated blood mononuclear cells with the nanoparticles having the immobilized peptide-presenting MHC molecules, the T-lymphocytes binding to the T-cell epitope of the immobilized peptide-presenting MHC molecules, and
d) removing the nanoparticles having the T-lymphocytes bound to the immobilized peptide-presenting MHC molecules from the unbound peripheral mononuclear cells.

109-113. (canceled)

114. A method for at least one of priming and restimulating a CD4⁺-T- or CD8⁺-T-lymphocyte reaction in vitro, which comprises
a) identifying a T-cell epitope as claimed in claim 1 and determining its amino acid sequence,
b) preparing a nucleic acid coding for a peptide having the amino acid sequence of the T-cell epitope,
c) introducing the nucleic acid prepared in step (b) into a suitable vector,
d) introducing the vector obtained in step (c) into dendritic cells isolated, if appropriate, from cultivated peripheral blood mononuclear cells,
e) propagating the dendritic cells obtained in step (d), which have the vector, in vitro, and
f) stimulating at least one of autologous CD4⁺- and CD8⁺-cells in vitro using the dendritic cells obtained in step (d) or (e).

115. A nanoparticle, comprising on the surface at least one receptor unit.

116-121. (canceled)

122. A nanoparticle having an immobilized MHC molecule, where the MHC molecule comprises a first and a second chain and the MHC molecule is immobilized on the nanoparticle surface by binding of a first functional group present in the first chain to a second functional group present on the nanoparticle surface or by binding of the first functional group present in the first chain to the second functional group present on the nanoparticle surface and binding of a third functional group present in the second chain to a fourth functional group present on the nanoparticle surface.

123. A nanoparticle having a peptide-presenting MHC molecule immobilized on the nanoparticle surface, where the peptide-presenting MHC molecule comprises a first chain, a second chain and a peptide of 8 to 24 amino acids and the MHC molecule is immobilized on the nanoparticle surface by binding of a first functional group present in the first chain to a second functional group present on the nanoparticle surface or by binding of the first functional group present in the first chain to the second functional group present on the nanoparticle surface and binding of a third functional group present in the second chain to a fourth functional group present on the nanoparticle surface.

124-127. (canceled)

128. A peptide vaccine which comprises at least one peptide-presenting MHC molecule preparable as claimed in claim 55 and/or which comprises at least one protein antigen which contains a T-cell epitope identifiable by the method as claimed in claim 1.

129-131. (canceled)

132. A kit for at least one of identifying and detecting T-cell epitopes of a protein antigen in vitro, comprising a container with a suspension of nanoparticles having an immobilized MHC molecule as claimed in claim 122 or a container with a suspension of nanoparticles having an immobilized first chain of an MHC molecule as claimed in claim 115 and a container with a lyophilizate of a second chain.

133. A method for at least one of identifying and detecting T-cell epitopes of a protein antigen in vitro wherein a nanoparticle as claimed in claim 115 is used.

134. A method for preparing a peptide vaccine wherein a nanoparticle as claimed in claim 115 is used.

135. A method for at least one of enriching and isolating specific CD4⁺-T-lymphocytes or CD8⁺-T-lymphocytes in vitro wherein a nanoparticle as claimed in claim 115 is used.

136. A method for at least one of priming and restimulating a CD4⁺- and/or CD8⁺-T-lymphocyte reaction in vitro wherein a nanoparticle as claimed in claim 115 is used.

137. A method for the active immunization of an animal or human organism against a protein antigen wherein a peptide vaccine as claimed in claim 128 is used.

138. The method of claim 55 which further comprises at least one of the following additional steps:
a) preparing, by genetic engineering or chemical synthesis, suitable amounts of a peptide based on the determined amino acid sequence of the peptide fragment,
b) preparing, by genetic engineering or chemical synthesis, suitable amounts of the first and second chains,
c) preparing suitable amounts of peptide-presenting MHC molecules by joint incubation of the first chain, the second chain and the peptide prepared, and
d) preparing a peptide vaccine in the form of a lyophilizate or an aqueous colloidal solution or suspension of the peptide-presenting MHC molecules.
